(19) United States
(12) Patent Application Publication
(10) Pub. No.: US 2021/0045646 A1
(43) Pub. Date: Feb. 18, 2021

(54) SYSTEM AND METHOD FOR IDENTIFYING A FOCAL AREA OF FUNCTIONAL PATHOLOGY IN ANESTHETIZED SUBJECTS WITH NEUROLOGICAL DISORDERS

(71) Applicants: LOUISIANA TECH RESEARCH CORPORATION, Ruston, LA (US); THE UAB RESEARCH FOUNDATIN, Birmingham, AL (US)

(72) Inventors: Leonidas Iasemidis, Ruston, LA (US); Timothy N. Hutson, Ruston, LA (US); Omar A. Alamoudi, Ruston, LA (US); Sandipan B. Pati, Birmingham, AL (US)

(21) Appl. No.: 16/645,555

(22) PCT Filed: Sep. 14, 2018
(86) PCT No.: PCT/US18/51103

§ 371 Date: Mar. 9, 2020

Related U.S. Application Data
(60) Provisional application No. 62/558,580, filed on Sep. 14, 2017.

(51) Int. Cl. A61B 5/04; A61B 5/0478; A61B 5/00; A61B 5/048; G16H 50/30; G16H 50/20

(52) U.S. Cl. CPC A61B 5/04012; A61B 5/0478; A61B 5/7203; A61B 5/048; A61B 5/7246; A61B 5/7264; G16H 50/20; A61B 5/7282; A61B 5/4094; A61B 5/4839; A61B 5/4842; A61B 5/742; G16H 50/30; A61B 5/7275

(57) ABSTRACT

The present disclosure relates to identification of focal areas of abnormal interactions in a brain of an anesthetized subject. During the maintenance period of anesthesia and/or the post-emergence period of anesthesia, neurophysiological time series signals from two or more areas of the brain of the subject can be recorded. A system that includes a processor can receive these signals, estimate a percentage of time (POT) each of the two or more areas of the brain of the subject exhibits a maximum total effective inflow (TEI) of information, and localize one or more focal areas of abnormal brain interactions based on the POT of each of the at least two areas exhibiting maximum TEI.

### RELATED APPLICATIONS

This application claims the benefit of U.S. Provisional Application No. 62/558,580, filed Sep. 14, 2017, entitled “LOCALIZATION OF BRAIN'S FUNCTIONAL PATHOLOGY UNDER ANESTHESIA,” the entirety of which is hereby incorporated by reference for all purposes.

### GOVERNMENT SUPPORT

This invention was made with U.S. government support under OIA 1632891 awarded by the National Science Foundation. The government has certain rights in this invention.

### TECHNICAL FIELD

The present disclosure relates generally to an alternative, robust, and effective way to identify a focal area of functional pathology and, more specifically, to systems and methods that can identify the focal area of functional pathology in an anesthetized subject with a neurological disorder.

### BACKGROUND

Epilepsy is among the most common disorders of the nervous system, affecting 1-2% of the world's population. It is a unique paroxysmal disorder characterized by chronically recurrent disruptions of the brain's normal activity (seizures), resulting from excessive electrical discharges of abnormal groups of neurons (the epileptogenic focus). The poor quality of life and psychosocial functioning associated with epilepsy exacts an enormous toll on patients and their families. Epilepsy has a substantial impact on society because patients lose employment potential while incurring high bills for their medical care.

Despite many decades of research and the development of new antiepileptic drugs, a large number (30-40%) of patients suffer from inadequately controlled seizures or undesirable side effects from their medication. For these patients, seizures can be controlled by surgical treatment (e.g., resective epilepsy surgery) and/or neuromodulation (e.g., targeted electrical stimulation). However, these treatments are only effective in patients in which the epileptogenic focus can be localized with a high degree of confidence.

Traditionally, neuro-recording methods (e.g., long-term electroencephalographic (EEG) recordings, magnetic resonance imaging (MRI), positron emission tomography (PET), subtraction ictal single photon emission computed tomography (SPECT) co-registered with MRI (SISCOM), and magnetoencephalography (MEG)) have been used to identify the epileptogenic focus; however, such neuro-recording studies, when they are conducted, are often inconclusive or negative since seizures typically occur unpredictably and without warning, and interictal (seizure-free) periods may not exhibit epileptiform abnormalities (e.g., interictal spikes). Localization of the epileptogenic focus is traditionally performed at seizures onset, but this localization technique also remains limited due to the involved ambiguities in subsequent assessment of the seizure onset zone, patient discomfort because of the requirement of tapering his/her anti-epileptic medication and having his/her typical seizures during long (days) stay as inpatient in the limited number of available epilepsy monitoring units (EMUs), and the involved high cost of this procedure. Accordingly, development of alternative, robust, and effective ways to localize the focal areas from interictal periods can allow more epilepsy patients to have their seizures controlled by surgical treatment and/or neuromodulation.

### SUMMARY

The present disclosure relates generally to an alternative, robust, and effective way to identify a focal area of functional pathology (e.g., during interictal periods) and, more specifically, to systems and methods that can identify a focal area of functional pathology in an anesthetized subject with a neurological disorder. The identification of the focal area of abnormal brain interactions can occur during the maintenance period of anesthesia and/or the post-emergence period of anesthesia. Notably, the subject need not be tapered from any drugs taken to control the neurological disorder.

In one aspect, the present disclosure can include a system that identifies a focal area of abnormal brain interactions in an anesthetized subject during the maintenance period of anesthesia and/or the post-emergence period of anesthesia. The system can include a non-transitory memory storing computer-executable instructions and a processor that executes the computer-executable instructions to at least: receive neurophysiological time series signals from two or more areas of a brain of a subject under influence of an anesthetic agent; estimate, from the signals, a percentage of time (POT) each of the two or more areas of the brain of the subject exhibits a maximum total effective inflow (TEI); and localize one or more focal areas of abnormal brain interactions based on the POT of each of the at least two areas exhibiting the maximum TEI.

In another aspect, the present disclosure can include a method for identifying a focal area of abnormal brain interactions in an anesthetized subject during the maintenance period of anesthesia and/or the post-emergence period of anesthesia. The method can include steps that can be performed by a system that includes a processor. The steps can include: receiving neurophysiological time series signals from two or more areas of a brain of a subject under influence of an anesthetic agent; estimating from the signals a percentage of time (POT) each of the two or more areas of the brain of the subject exhibits maximum total effective inflow (TEI); and localizing one or more focal areas of abnormal brain interactions based on the POT of the areas that exhibit maximum TEI.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a schematic block diagram showing a system that can identify a focal area of abnormal brain interactions in an anesthetized subject in accordance with an aspect of the present disclosure;

FIG. 2 is a schematic block diagram showing an information inflow determination unit that can be part of the system shown in FIG. 1;

FIG. 3 is a schematic block diagram showing a focal area identification unit that can be part of the system shown in FIG. 1;

FIG. 4 is a process flow diagram illustrating a method for identifying a focal area of abnormal brain interactions in a subject in an anesthetized subject in accordance with another aspect of the present disclosure;

FIG. 5 is a process flow diagram illustrating an example focal area identification method that can be implemented within the method of FIG. 4;

FIGS. 6 and 7 each include plots showing the percentage of time (POT) that each of 102 brain sites of a patient 1 hour post-surgical electrode implantation (patient P1) exhibited maximum effective inflow in two different frequency bands;

FIG. 8 is a plot showing brain sites that exhibited maximum POT per hour over time in a patient right after electrode implantation; and

FIG. 9 is a plot showing the statistical significance of the identified pathological site (focal area) over time denoting the period that the focus is best identified by POT.

### DETAILED DESCRIPTION

### I. Definitions

In the context of the present disclosure, the singular forms “a,” “an” and “the” can also include the plural forms, unless the context clearly indicates otherwise.

As used herein, the terms “comprises” and/or “comprising” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. can describe various elements, but these elements should not be limited by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the term “anesthesia” can refer to a medically-induced and reversible coma characterized by physiological any electrophysiological traits associated with coma and maintaining the stability of homeostatic processes. Anesthesia includes three periods: (1) the induction period, (2) the maintenance period, and (3) the emergence period.

As used herein, the term “induction period” can refer to a period of anesthesia where consciousness is lost. Physiologically, the subject is calm and easily aroused, with eyes generally closed. Movements are often defensive or purposeless, accompanied by incoherent speech, and euphoria or dysphoria. As dosage of an anesthetic drug begins to increase, breathing support is required following apnea. Heart rate typically increases during this period. Electrophysiologically, electroencephalogram (EEG) power in the beta range of the spectrum (13-25 Hz) increases during the induction period.

As used herein, the term “maintenance period” can refer to a period of anesthesia where the subject is unconscious. Physiologically, muscle tone is lost, cardiovascular changes are used to monitor depth of this period. Electrophysiologically, beta activity (13-25 Hz) decreases initially, followed by an increase in alpha (8-12 Hz) and delta (0-4 Hz) activity. The maintenance period EEG is characterized by low and flat activity, followed by isoelectric activity.

As used herein, the term “emergence period” can refer to a period of anesthesia where the subject begins to regain consciousness. Physiologically, muscle tone returns and cardiovascular functions are more self-sufficient. Heart rate and blood pressure typically increase. The patient is often drowsy or still unconscious. Electrophysiologically, burst activity returns, beta activity returns to normal, as does the activity in other frequency bands.

As used herein, the term “anesthetic agent” can refer to any pharmaceutical agent that inhibits one or more neurotransmitters in the cortex that promote arousal. The anesthetic agent can promote a decrease in cortical activity and fragmentation of communication between deep brain structures and cortical structures. The anesthetic agent can be delivered intravenously; examples of such anesthetic agents include barbiturates, benzodiazepines, etomidate, ketamine, and propofol.

As used herein, the term “focal area” can refer to an area of the brain where abnormal brain interactions are initiated or localized. The focal area may also be referred to as a hot spot. The term focal area can include a single focal area, bifocal areas (e.g., two focal areas), or multifocal areas (e.g., more than two focal areas).

As used herein, the term “brain interactions” can refer to bidirectional relations between one or more electrical potentials, magnetic field potentials, and/or currents and/or other physiological signals (e.g., blood flow, neurotransmitter exchange, metabolism, etc.) generated by one or more regions or areas of the brain and detectable by a neuro-recording modality. In an example, the brain interactions can be abnormal brain interactions between regions of the brain that can be indicative of abnormal brain activity.

As used herein, the term “neurological disorder” can refer to a disorder of the nervous system causing functional pathology and characterized by abnormal brain activity. Examples of neurological disorders include, but are not limited to, paroxysmal neurological disorders (e.g., epilepsy, multiple sclerosis, encephalitis, traumatic brain injury, stroke, trigeminal neuralgia, etc.), conditions that include a lack of awareness, conditions that include a lack of cognition, neurodegenerative diseases, psychiatric disorders, psychological disorders, obesity disorders, apnea disorders, Autism spectrum disorders, and Alzheimer's disease.

As used herein, the term “neurorecording modality” can refer to a recording modality that can record time series data corresponding to biosignals emitted from, or associated with, one or more regions of the brain. In some instances, the neuro-recording modality can include two or more recording channels (e.g., corresponding to two or more regions of the brain). Examples of recording modalities can include, but are not limited to, electroencephalogram (EEG) (e.g., invasive EEG (intracranial iEEG) or noninvasive (scalp EEG)), magnetoencephlogram (MEG), thermal imaging, positron emission tomography (PET), Single Photon Emission Computed Tomography (SPECT), and functional magnetic resonance imaging (fMRI).

As used herein, the term “information inflow” can refer to a characteristic of a brain region that reflects the flow of information to the brain region from other brain regions. Information inflow can be based on directional connectivity between the brain region and at least one other brain region.

As used herein, the term “average information inflow” can refer to the information inflow to certain brain region that is averaged over a certain time period.

As used herein, the terms “directed connectivity” and/or “directional connectivity” can refer to an estimate of functional connectivity from a brain region and at least one other brain region. In one example, directional connectivity can be derived from time series signals, recorded from a neuro-recording modality from multiple brain regions, via multivariate autoregressive modeling of the time series signals. In another example, directional connectivity can be derived from a time series signal that contains activity from a plurality of brain regions and is recorded from a neuro-recording modality, via autoregressive modeling of the time series signal.

As used herein, the term “treatment plan” can refer to a definition of medical care to be given to a patient for a neurological disorder related to the focal area. The treatment plan can involve surgery and/or neuromodulation (e.g., targeted drug release, electrical or magnetic stimulation, in situ administration of a heat/cool agent, etc.).

As used herein, the term “anesthetized subject” can refer to a subject during the maintenance period of anesthesia and/or the post-emergence period of anesthesia.

As used herein, the term “subject” can refer to any warm-blooded organism including, but not limited to, a human being, a pig, a rat, a mouse, a dog, a cat, a goat, a sheep, a horse, a monkey, an ape, a rabbit, a cow, etc. The terms “subject” and “patient” can be used interchangeably herein.

### II. Overview

Localization of the focal area of abnormal brain interactions characteristic of functional pathology remains a difficult task due to ambiguities in assessment of the location of the focus, patient discomfort due to tapering of his/her medicine in order to manifest his/her typical seizures for focus localization, and large cost of the involved procedure. Accordingly, the present disclosure relates generally to an alternative, robust, and effective way to identify a focal area of abnormal brain interactions that has fewer ambiguities, does not require tapering of patient medication and manifestation of his/her seizures, and a lower cost. The identification of the focus of abnormal brain interactions can be performed on an anesthetized subject. The anesthetized subject need not be tapered from the medicine that controls the abnormal brain interactions. Additionally, the localization results have fewer ambiguities due to the compartmentalization of brain networks while the brain is under anesthesia. Moreover, the costs are reduced because long stays in an epilepsy monitoring unit (EMU) of the hospital are not required as the patient is not required to manifest his/her typical seizures during his stay in the hospital. Advantageously, the present disclosure can further facilitate advances in the diagnosis and treatment of neurological disorders characterized by abnormal brain interactions by, for example: complementing the current clinical practice diagnostic procedures for standard-of-care focus identification (e.g. localization of the focus from the very first time period of a patient's stay in the EMU and before tapering of his/her medication); quickly and accurately determining patients that may require invasive monitoring if focus localization from non-invasive (e.g. scalp EEG or MEG) recordings is ambiguous; identifying the location of the focal area from invasive recordings of signals in patients with drug-resistant neurological disorders; improving the treatment of neurological disorders by better delineating the extent of surgical resection or identifying the target for implantable stimulators, drug infusion, or other neuromodulation devices; and monitoring the dynamics of the focal area over time to provide insights into the mechanism of generation of the abnormal brain interactions and thus increase the accuracy of prediction of crises (e.g., seizures) by intelligent prediction devices.

More specifically, the present disclosure relates to systems and methods that can identify a focal area of abnormal brain interactions in an anesthetized subject (while the anesthetized subject is in the maintenance period of anesthesia and/or the post-emergence period of anesthesia). In some instances, the systems and methods described herein can employ a computer-implemented technique that can identify a focal area (e.g., one or more epileptogenic foci) from neurophysiological time series signals recorded from the brain of the anesthetized patient. As described in more detail below, the present disclosure employs measures of directional connectivity to estimate and quantify the strength of information flow between different brain regions to determine the focal area. The average information inflow to each region from other regions can then be used to identify the focal area as the region exhibiting the greatest number of instances of maximum average inflow across regions.

### III. Systems

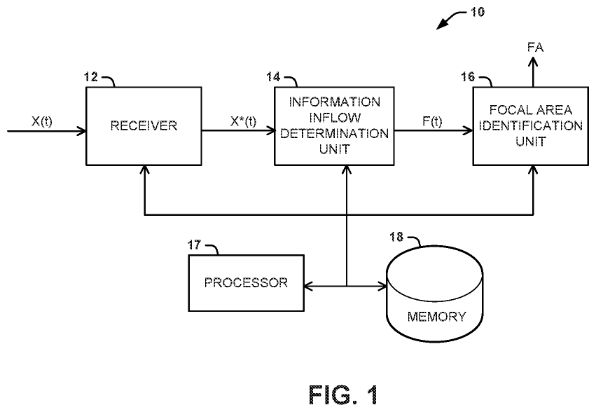

One aspect of the present disclosure can include a system 10 (shown in FIG. 1) that can identify a focal area (FA) of abnormal brain interactions from time series signals (X(t)) recorded from areas of the brain of an anesthetized subject when the patient is in the maintenance period of anesthesia and/or the post-emergence period of anesthesia. As an example, the time series signals (X(t)) can be recorded twelve hours or less after administration of an anesthetic agent, entry into the maintenance period of anesthesia, and/or entry into the post-emergence period of anesthesia. As another example, the time series signals (X(t)) can be recorded six hours or less after administration of an anesthetic agent, entry into the maintenance period of anesthesia, and/or entry into the post-emergence period of anesthesia. In a further example, the time series signals (X(t)) can be recorded four hours or less after administration of an anesthetic agent, entry into the maintenance period of anesthesia, and/or entry into the post-emergence period of anesthesia.

The term focal area can refer to one or more areas in the brain where abnormal brain interactions are initiated or localized. The focal area can be associated with, for example, an epileptic seizure disorder, a paroxysmal neurological disorder, a stroke, an autism spectrum disorder, a psychological disorder, a traumatic brain injury, an obesity disorder, an apnea disorder, a condition comprising a lack of awareness, a neurodegenerative disease, or the like. The time series signals (X(t)), which include time series data corresponding to biosignals emitted from, or associated with, one or more regions of the brain, can be recorded by a neuro-recording modality (e.g., electroencephalogram (EEG), magnetoencephlogram (MEG), thermal imaging, functional magnetic resonance imaging (fMRI), positron emission tomography (PET), Single Photon Emission Computed Tomography (SPECT), or the like).

It has been observed that the focal area becomes more statistically significant with respect to Total Effective Inflow (TEI) when the patient is in the maintenance period of anesthesia and/or the post-emergence period of anesthesia. Using time series data recorded from an anesthetized subject's brain that exhibits the stronger statistically significant focal area, the system 10 can provide an alternative, robust, and effective way to identify the focal area of abnormal brain interactions that has fewer ambiguities, does not require tapering from drugs taken to control the abnormal brain interactions, and at a lower cost. The anesthetized subject need not be tapered from the medicine to control the abnormal brain interactions. The localization results have fewer ambiguities due to the compartmentalization of brain networks while the brain is under anesthesia. Moreover, the costs are reduced because long stays in the hospital are not required.

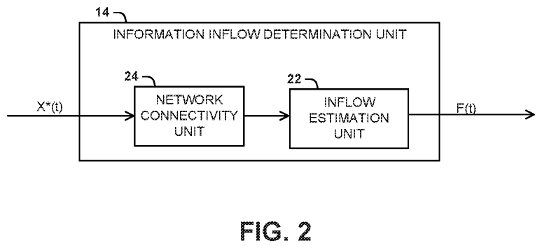

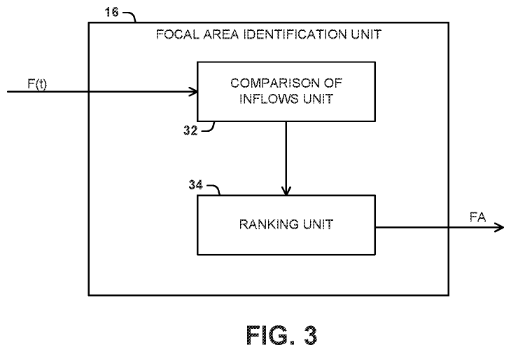

FIG. 1, as well as associated FIGS. 2-3, are schematically illustrated as block diagrams with the different blocks representing different components. The functions of one or more of the components can be implemented by computer program instructions. These computer program instructions can be provided to a processor of a general purpose computer, special purpose computer, and/or other programmable data processing apparatus to produce a machine, such that the instructions, which execute via the processor of the computer and/or other programmable data processing apparatus, create a mechanism for implementing the functions of the components specified in the block diagrams.

These computer program instructions can also be stored in a non-transitory computer-readable memory that can direct a computer or other programmable data processing apparatus to function in a particular manner, such that the instructions stored in the non-transitory computer-readable memory produce an article of manufacture including instructions, which implement the function specified in the block diagrams and associated description.

The computer program instructions can also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions of the components specified in the block diagrams and the associated description.

Accordingly, the system 10 described herein can be embodied at least in part in hardware and/or in software (including firmware, resident software, micro-code, etc.). Furthermore, aspects of the system 10 can take the form of a computer program product on a computer-usable or computer-readable storage medium having computer-usable or computer-readable program code embodied in the medium for use by or in connection with an instruction execution system. A computer-usable or computer-readable medium can be any non-transitory medium that is not a transitory signal and can contain or store the program for use by or in connection with the instruction or execution of a system, apparatus, or device. The computer-usable or computer-readable medium can be, for example but not limited to, an electronic, magnetic, optical, electromagnetic, infrared, or semiconductor system, apparatus or device. More specific examples (a non-exhaustive list) of the computer-readable medium can include the following: a portable computer diskette; a random access memory; a read-only memory; an erasable programmable read-only memory (or Flash memory); and a portable compact disc read-only memory.

As shown in FIG. 1, the system 10 configured to identify a FA of abnormal brain interactions in an anesthetized subject. Time series signals (X(t)) can be recorded from the brain of the anesthetized subject, who can be in the maintenance period of anesthesia and/or the post-emergence period of anesthesia The system 10 can identify the FA in an invasive manner and/or a non-invasive manner (based on how the time series data was recorded) from the time series data (X(t)). As noted above, identification of the FA advantageously can facilitate the development of future diagnostics and treatments (e.g., better targeted treatments) for the abnormal brain interactions.

In one example, the system 10 can be utilized in epileptic patients to identify the epileptogenic focus (e.g., in an invasive manner and/or a non-invasive manner). Identification of the epileptogenic focus can facilitate advances in the diagnosis and treatment of epilepsy by, for example: complementing the current clinical practice diagnostic procedures for standard-of-care focus identification; quickly determining patients that may require invasive monitoring; quickly identifying the location of the epileptogenic focus in patients with drug-resistant focal epilepsies; improving the treatment of epilepsy by better delineating the extent of surgical resection or target for implantable stimulators and drug infusion devices; studying the dynamics of the epileptogenic focus over time to provide insights into the mechanism of epileptogenesis; developing biomarkers and surrogate markers for the presence of epileptogenic networks in high risk patients who are susceptible of developing epilepsy in the future; and creating an outpatient setting for focus localization from routine tests.

The system 10 can include components including at least a receiver 12, an information inflow determination unit 14, and a focal area identification unit 16. One or more of the components can include instructions that are stored in a non-transitory memory 18 and executed by a processor 17. Each of the components can be in a communicative relationship with one or more of the other components, the processor 17, and/or the non-transitory memory 18 (e.g., via a direct or indirect electrical, electromagnetic, optical, or other type of wired or wireless communication) such that an action from the respective component causes an effect on one or more of the other components.

The receiver 12 can be configured to receive time series data (X(t)) recorded from a plurality of regions in a brain of the anesthetized subject. For example, each of the plurality of regions can correspond to a position of a unique recording electrode and/or a reconstructed source of brain activity. In one example, the time series data (X(t)) can be recorded by a neuro-recording modality that includes a plurality of recording channels (e.g., corresponding to the plurality of regions of the brain) at different places in space (e.g., spatial recording positions).

In some instances, the time series data (X(t)) can be an n-dimensional time series vector representation of different signals corresponding to n different spatial locations:

X(t)→(X₁(t), X₂(t), . . . , Xₙ(t))′, Equation 1

where n corresponds to a total number of different spatial locations (corresponding to regions of the brain) where time series signals were recorded, and each vector component Xᵢ(t) denotes the signal recorded at the i^{th} recording site.

The input time series data (X(t)) can include raw time series signals obtained from or generated by a neuro-recording modality. In one example, the neuro-recording modality can be EEG, and the different vector components of the input time series data (X(t)) can correspond to different locations of one or more EEG sensors. In another example, the neuro-recording modality can be MEG, where the receiver 12 can preprocess the raw input time series data (X(t)) into preprocessed time series data (X*(t)). The term “preprocessed time series data (X*(t))” can refer to input to the information inflow determination unit 14 to prevent confusion with the time series data (X(t)) that is input to the receiver 12. In another example, the neurorecording modality can be an fMRI, PET or SPECT unit that records physiological signals from the brain over time.

The preprocessed time series data (X*(t)) can include processed signals that are generated from electromagnetic sources in brain regions estimated via the fitting of the raw data X(t) by a brain-source model (e.g., via a type of inverse modeling that may include weighted minimum norm estimates (wMNE), linearly constrained minimum variance (LCMV) beamformers, low resolution electrical tomography (LORETA), etc.). Such a brain-source model can be used to estimate the position and orientation of possible sources (e.g., current dipoles) in the brain that can explain the observed raw signals X(t) over time. For example, the estimates of the position and orientation of such brain-sources can be assigned by three-dimensional MRI images of the subject.

The receiver 12 can provide the time series data (X(t)) and/or the preprocessed data (X*(t)) to an information inflow determination unit 14. For example, the receiver 12 can divide the time series data (X(t)) into a series of time epochs so that the information inflow determination unit 14 can perform its analysis for the different epochs (e.g., each epoch corresponds to a time period in which the maximum information inflow can be determined). The epochs can be non-overlapping or random (e.g., containing one or more overlapping portions). For simplicity of illustration and explanation, the receiver 12 is illustrated as providing the preprocessed data (X*(t)) to the information inflow determination unit 14. Although the preprocessed time series data (X*(t)) is referenced herein, it will be appreciated that the receiver 12 need not perform the preprocessing step and can provide the time series data (X(t)) to the information inflow determination unit 14. The preprocessed time series data (X*(t)) possesses vector properties similar to those as defined for the time series data (X(t)).

The information inflow determination unit 14 can be configured to execute a localization technique on the preprocessed time series data (X*(t)) that is different from traditional FA localization techniques. Traditional techniques process the information outflow from the FA during abnormal brain activity. In contrast, the information inflow determination unit 14 is configured to determine information inflow associated with each of the brain regions (F(t)) based on the time series data (X(t)) or the preprocessed time series data (X*(t)).

The information inflow associated with a particular brain region can be determined from inflows to the particular brain region from one or more of the other regions of the brain. To shorten the associated processing time, the information inflow associated with a particular region can also be determined from inflows from a portion of the plurality of regions of the brain that is less than all of the regions (e.g., a finite number of neighboring regions to the particular region).

As shown in FIG. 2, the information inflow determination unit 14 can include an inflow estimation unit 22 and a network connectivity unit 24. The inflow estimation unit 22 can be configured to simulate a model representation of the time series data (X(t)) or the preprocessed time series data (X*(t)). The inflow estimation unit 22 can estimate a model representation of the preprocessed time series data (X*(t)). The model representation can be an autoregressive model that allows the preprocessed time series data (X*(t)). For example, the model representation can be a vector autoregressive model (VAR) or a multivariate autoregressive model (MVAR).

In one example, the autoregressive model VAR(p) can be constructed of an order p (where p is a pre-determined value—like an autocorrelation of the time series data—or an optimally determined value). For example, an autoregressive model can be expressed as:

$$VAR ( p ) = {\sum }_{τ = 1}^{p} B ( τ ) X * ( t - τ ) + ɛ ( t ) , \qquad Equation 2$$

where B(π) represents the n×n coefficient matrices of the model with residuals ϵ(t) ideally following a multivariate Gaussian white noise process.

The network connectivity unit 24 can be configured to quantify the network connectivity in the frequency domain based on the model representation of the time series data (X(t)) or the preprocessed time series data (X*(t)). The network connectivity unit 24 can be configured to quantify the network connectivity (interaction between brain regions) in the frequency domain based on the model representation (e.g., VAR(p)). The term “frequency domain” can refer to the behavior of the biological signal rather than the time period of the recording (e.g., high frequency relates to a rapidly changing signal and low frequency relates to a slowly changing signal). The interaction between brain regions can be estimated by one or more of the following: a directional measure (e.g., capturing the directionality of the flows); a non-directional measure (e.g., not capturing the directionality of the flows); a direct measure (e.g., capturing direct interactions); and/or a non-direct measure (e.g., capturing direct and indirect interactions).

In one example, the network connectivity unit 24 can apply a measure of the generalized partial directional coherence (GPDC) to capture the interactions between the various brain regions (which can be bidirectional). GPDC is a normalized version of partial directional coherence (PDC) that has been used in many applications for the study of brain dynamics. GPDC provides a measure for the direct linear influence of region Xⱼ on region Xᵢ at frequency f conditioned by the rest of the signal variables:

$$GPDCij ( f ) = \frac{\frac{\vert Bij ( f ) \vert}{σ ij}}{\sqrt{\frac{{\vert Bjk ( f ) \vert}^{2}}{{σ}^{2} k k}}} , \qquad Equation 3$$

where σᵢⱼ is obtained from the covariance matrix of ϵ(t), S=[σᵢⱼ]ᵢ, ⱼ₌₁₋ₙ, and Bᵢⱼ(f) is the (i,j)^{th} element of the matrix

$$B ( f ) = I - {\sum }_{τ - 1}^{p} B ( τ ) {e}^{- i 2 π f τ} ,$$

where I is the n×n identity matrix.

The network connectivity unit 24 can estimate the average directional connectivity index between nodes (corresponding to information flows for the regions of the brain) based on the quantification of the network connectivity (e.g., from the GPDC) over a given frequency range (f₁, f₂) Hz. The information inflow to a brain region A from another brain region B can be the portion of information content (e.g., behavior, content, etc.) of the brain region A that is due to or can be explained from its directional connectivity to brain region B. The directional connectivity from brain region B to brain region A can be the estimate of functional connectivity from brain region B to brain region A from appropriate mathematical analysis of the time series signals recorded in regions A and B (e.g., via multivariate autoregressive modeling of the involved time series signals).

The information inflow to a particular region of the brain can be determined by a weighted sum of the information inflows from the rest of the nodes j. For example, assuming a simple sum:

$$I n D i = {{\sum }_{j = 1 , j \neq i}}^{n} ( GPDCj \to i ( f ) ) . \qquad Equation 4$$

To increase the accuracy associated with the identification of the FA, the statistically significant information inflows (e.g., p<0.05) between the regions can be counted in the determination of information inflow to each region. One way the statistical significance of an information inflow can be evaluated is via a surrogate data scheme. For example, estimation of information inflows from data that is surrogate for real signals for which inter-sample dependencies have been artificially randomized. In an anesthetized patient, more inflows are statistically significant.

The average information inflows for all or a subset of the nodes (F(t)) can be provided by the information inflow determination unit 14 to the focal area identification unit 16. The focal area identification unit 16 can be configured to identify the FA of the abnormal brain interactions as one or more of the plurality of regions having a maximum information inflow (e.g., by comparing the information inflow of each of the plurality of regions).

As shown in FIG. 3, the focal area identification unit 16 can include a comparison unit 32 and a ranking unit 34. The comparison unit 32 can compare the value of the information inflow of each of the regions during one or more time periods (e.g., epochs) to determine the region exhibiting a maximum inflow value. The comparison unit 32 can compare the values of information flow by employing a statistical test associated with a property of information inflow (e.g., an outlier detection test). One example of an outlier detection test is a test of Grubb's outliers at a significance level α. In other words, the comparison unit 32 can estimate a percentage of time (POT) areas of the brain of the subject exhibits a maximum total effective inflow (TED.

The ranking unit 34 can determine the region most frequently exhibiting a maximum information inflow value (e.g., based on the POT the areas of the brain of the subject exhibit the maximum TEI) and identify this region as the FA of abnormal brain activity. For example, the ranking unit 34 can construct a histogram of the information inflow associated with the plurality of regions, and then identify the region with the maximum information flow based on the histogram. The region with the maximum information flow can be identified as the FA. For the identification of the FA, the region of the brain with the maximum information inflow can be estimated over at least a portion of the plurality of brain regions. The identification of the FA can also be based on comparison between ipsilateral and contralateral brain regions to determine the maximum inflow.

### IV. Methods

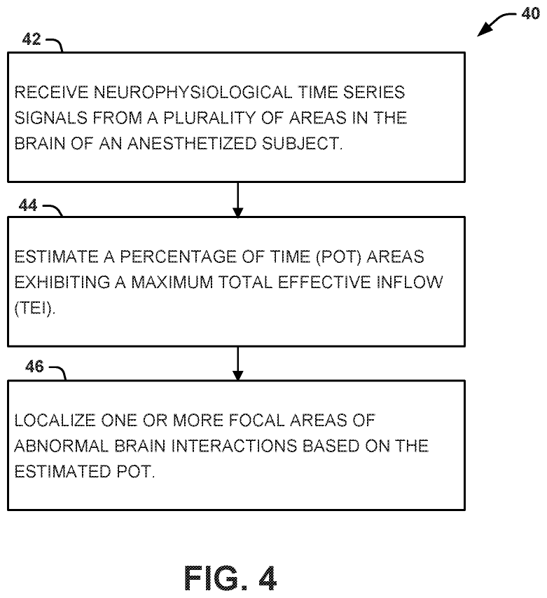

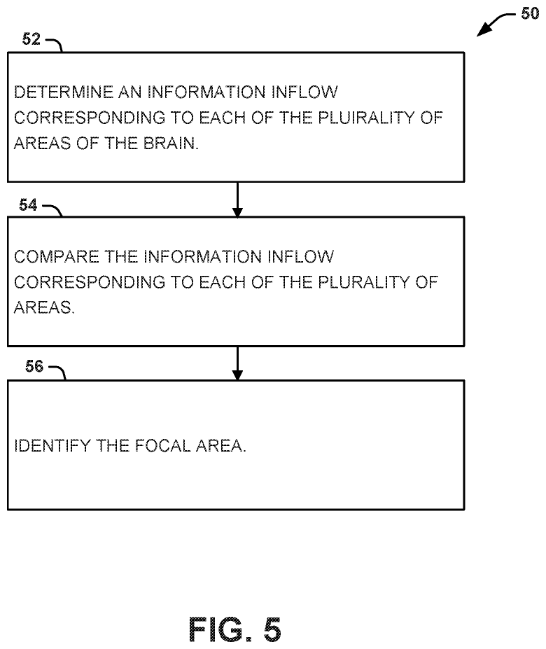

Another aspect of the present disclosure can include methods that can be used to identify one or more focal areas (FAs) of abnormal brain interactions in an anesthetized subject from time series data recorded during the resting period. Notably, the anesthetized subject can be in the maintenance period of anesthesia and/or the post-emergence period of anesthesia. An example of a method 40 that can identify the FA is shown in FIG. 4. FIG. 5 shows an example focal area identification method 50 that can be implemented within the method 40 of FIG. 4.

The methods 40 and 50 of FIGS. 4 and 5, respectively, are illustrated as process flow diagrams with flowchart illustrations. For purposes of simplicity, the methods 40 and 50 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 40 and 50.

One or more blocks of the respective flowchart illustrations, and combinations of blocks in the block flowchart illustrations, can be implemented by computer program instructions. These computer program instructions can be stored in memory and provided to a processor of a general purpose computer, special purpose computer, and/or other programmable data processing apparatus to produce a machine, such that the instructions, which execute via the processor of the computer and/or other programmable data processing apparatus, create mechanisms for implementing the steps/acts specified in the flowchart blocks and/or the associated description. In other words, the steps/acts can be implemented by a system comprising a processor that can access the computer-executable instructions that are stored in a non-transitory memory.

The methods 40 and 50 of the present disclosure may be embodied in hardware and/or in software (including firmware, resident software, micro-code, etc.). Furthermore, aspects of the present disclosure may take the form of a computer program product on a computer-usable or computer-readable storage medium having computer-usable or computer-readable program code embodied in the medium for use by or in connection with an instruction execution system. A computer-usable or computer-readable medium may be any non-transitory medium that can contain or store the program for use by or in connection with the instruction or execution of a system, apparatus, or device.

Referring to FIG. 4, an aspect of the present disclosure can include a method 40 for identifying a focal area of abnormal brain interactions in an anesthetized subject (in a maintenance period and/or a post-emergence period) from time series data recorded from at least two regions of the brain. The time series data can be recorded by a neuro-recording modality (e.g., EEG, MEG, thermal imaging, PET, fMRI, etc.).

At 42, time series data (X(t)) from a plurality of areas (or “regions”) in the brain of the anesthetized subject can be received (e.g., by receiver 12). As an example, the time series signals (X(t)) can be recorded twelve hours or less after administration of an anesthetic agent, entry into the maintenance period of anesthesia, and/or entry into the post-emergence period of anesthesia. As another example, the time series signals (X(t)) can be recorded six hours or less after administration of an anesthetic agent, entry into the maintenance period of anesthesia, and/or entry into the post-emergence period of anesthesia. In a further example, the time series signals (X(t)) can be recorded four hours or less after administration of an anesthetic agent, entry into the maintenance period of anesthesia, and/or entry into the post-emergence period of anesthesia.

For example, each of the plurality of regions represented by the time series data can correspond to a position of a unique recording electrode and/or a reconstructed source of brain activity. While the time series data (X(t)) recorded during the resting period can include abnormal activity (e.g., epileptiform activity), the abnormal activity is not required within the time series signal (X(t)). In other words, the time series data (X(t)) can include entirely normal brain activity. In some instances, the time series data (X(t)) can be an n-dimensional time series vector representation of different signals corresponding to n different spatial locations (e.g., X(t)→(X₁(t), X₂(t), . . . , Xₙ(t)′). The input time series data (X(t)) can include raw time series signals obtained from or generated by a neuro-recording modality.

At 44, a percentage of time (POT) areas exhibit a maximum total effective inflow (TEI) is estimated (e.g., by the information inflow determination unit). At 46, one or more focal areas of abnormal brain interactions are localized (e.g., by the focal area identification unit 16) based on the POT. The identified one or more focal areas can be associated with at least one of an epileptic seizure disorder, a paroxysmal neurological disorder, a stroke, an autism spectrum disorder, a psychological disorder, a traumatic brain injury, an obesity disorder, an apnea disorder, a condition comprising a lack of awareness, and a neurodegenerative disease. A treatment plan can be developed for the abnormal brain interactions based on the one or more focal areas. The treatment plan can include surgery or stimulation (electrical or magnetic).

FIG. 5 shows a method 50 that can be used within the method 40 to determine the TEI and the POT. At 52, an information inflow corresponding to each of the plurality of areas of the brain (or “regions”) can be determined (e.g., by inflow estimation unit 22 of information inflow determination unit 14) based on the time series data. In some instances, the inflow can be determined for the higher frequency bands of the signals. However, the information inflow can also be determined for the lower frequency bands of the signals. The information inflow associated with a particular brain region can be determined from inflows from one or more of the other regions of the brain. To speed up processing time, the information flow associated with a particular region can also be determined from inflows from a portion of the plurality of regions of the brain less than all of the regions (e.g., a finite number of neighboring regions to the particular region).

The information inflow can be determined based on a simulation and/or estimation of a model representation of the time series data (X(t)). The model representation can be an autoregressive model that allows the time series data (X*(t)). For example, the model representation can be a vector autoregressive model (VAR) or a multivariate autoregressive model (MVAR). In an example, the autoregressive model VAR(p) can be constructed of an order p (where p is determined based on an autocorrelation of the time series data (X(t))).

Network connectivity can be quantified based on the model representation of the time series data (X(t)). In one example, a measure of the generalized partial directional coherence (e.g., GPDC that provides a measure of direct linear influence of different regions on one another) can be applied to capture the interactions between the various brain regions. The average directional connectivity index between nodes (corresponding to information flows for the regions of the brain) can be based on the quantification of the network connectivity (e.g., from the GPDC) over a given frequency range (f₁, f₂) Hz.

At 54, the information inflow corresponding to each of the plurality of regions can be compared (e.g., by network connectivity unit 24 of information inflow determination unit 14). The information inflow to a particular region of the brain can be determined by averaging over all of the information inflows from the rest of the nodes. To increase the accuracy associated with the identification of the FA, only the statistically significant information inflows (e.g., p<0.05) between the regions can be counted in the determination of information inflow to each region. One way the statistical significance of an information inflow can be evaluated is via a surrogate data scheme. For example, estimation of information inflows from data that is surrogate for real signals for which inter-sample dependencies have been artificially randomized.

At 56, the FA can be identified (e.g., by focal area identification unit 16) as one of the identified regions exhibiting a maximum information inflow. The value of the information inflow of each of the regions during one or more time periods can be compared to values associated with other regions to determine the region exhibiting a maximum inflow value (e.g., based on a statistical test associated with a property of information inflow, such as an outlier detection test). The region most frequently exhibiting the maximum information inflow value can be identified as the FA of abnormal brain interactions (e.g., from a histogram of the information inflow associated with the plurality of regions). For the identification of the FA, the region of the brain with the maximum information inflow can be estimated over all of the plurality of brain regions. The identification of the FA can also be based on comparison between ipsilateral and contralateral brain regions to determine the maximum inflow. Identification of the FA can facilitate the development of diagnostics and treatments for the abnormal brain interactions.

### V. EXAMPLE

The following example is for the purpose of illustration only and are not intended to limit the scope of the appended claims.

This example shows that the effects of anesthesia are beneficial for epileptogenic focus localization. Individual brain regions are more distinct under the influence of anesthesia because most extemporaneous connections are not as active due to the suppressive nature of the anesthesia. Accordingly, this example shows a methodology for focus localization in a set of patients with temporal and frontal lobe epilepsy during their first hours of recovery from general anesthesia following the implantation of electroencephalogram (EEG) electrodes and before tapering of anti-epilepsy drugs (AEDs). The results suggest that it is possible to accurately localize the focus early on in the post-surgical electrode implantation period Π in patients with focal epilepsy (e.g., when the patient is in the maintenance period of anesthesia and/or the post-emergence period of anesthesia).

### Methods

A set of 11 patients with focal epilepsy were recruited at the U. Alabama Birmingham. Each of the 11 patients consented for participation in the study.

Depth electrodes were implanted into each of the 11 patients during a surgical procedure with the patient under general anesthesia (Propofol). The implantation of the depth electrodes was image-guided, robot-assisted (ROSA). Post-implantation patients were extubated and transferred to neuro ICU. The depth electrodes were connected to the Natus Xltek EEG machine and on average 120 EEG channel recording at 2 KHz was initiated (H period).

The Generalized Partial Direct Coherence (GPDC) was employed, a measure of directed causal interactions between brain sites over low (LFB: 0.1-50Hz) and high (HFB: 70-110 Hz) frequency bands and a step of 0.1 Hz. The GPDC values were generated from a 7th order, 120-dimensional multivariate autoregressive model fitted to 100 sec successive and non-overlapping EEG segments for 1 hour in the Π period. The percentage of time (POT) that each site showed the maximum cumulative incoming GPDC values from the other sites where then estimated (effective inflow of information). The identification of the focus is more sensitive when the analysis is implanted in higher frequency bands (70-110 Hz); however, the analysis is still possible with respect to low frequency bands below (0.1-50 Hz).

### Results

Results from the 11 patients are shown in Table 1, which provides clinically determined focal area compared to the focal area determined from application of the algorithm (during the post-emergence stage of anesthesia). As shown in Table 1, the algorithm was successful at identifying the focal area during the post-emergence stage of anesthesia in 10/11 patients.

TABLE 1
iEEG Localization during the Post-Emergence Period

| | Focal Area- | Focal Area- | Correct |
|---|---|---|---|
| Patient | Clinical | Algorithm | Localization? |
| PT 1 | Left Hippocampus | Left Posterior | YES |
| | | Hippocampus | |
| PT 2 | Right Hippocampus | Right Hippocampus | YES |
| PT 3 | Left Mesial | Left Lateral Frontal | YES |
| | Frontal/Anterior | | |
| | Cingulate | | |
| PT 4 | Right Mesial | Left and Right | YES |
| | Frontal | Mesial Frontal | |
| PT 5 | Right Hippocampus | Right Hippocampus | YES |
| | STG | | |
| PT 6 | Left Hippocampus | Left Hippocampus | YES |
| PT 7 | Insular-Parietal | Anterior Insular | YES |
| PT 8 | Left Hippocampus | Left Posterior | YES |
| | | Hippocampus | |
| PT 9 | Hippocampal | FT | NO |
| | Temporal Pole | (Frontotemporal?) | |
| PT 10 | Left Hippocampus | Left Posterior | YES |
| | | Hippocampus | |
| PT 11 | Left Hippocampus | Left Posterior | YES |
| | Orbitofrontal | Orbitofrontal | |

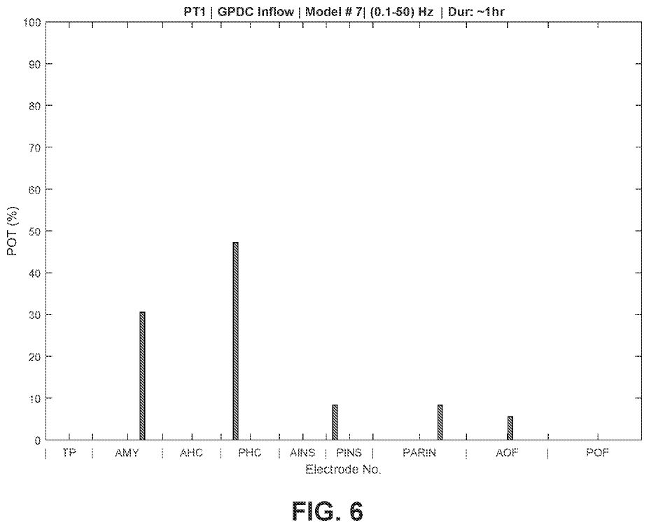

The results of the analysis per electrode site from one of the patients with left temporal epilepsy (using the above-described algorithm) are shown in FIGS. 6-9. The focal area results for the patient (PT₁) determined from the algorithm were consistent with the clinically determined focal area.

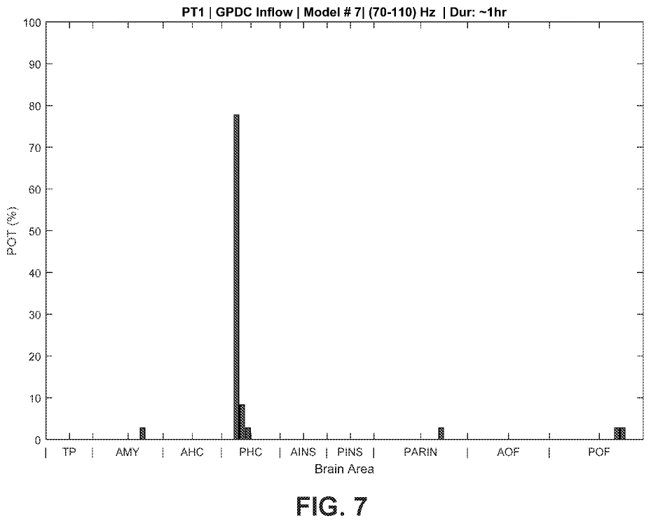

FIGS. 6 and 7 show the percentage of time that each of 120 brain sites of patient PT₁ exhibited maximum effective inflow per 10-second iEEG segment and over 1 hour post-surgical electrode implantation. While multiple sites are selected as candidate focal sites from GPDC analysis in the low frequency band (0.1-50 Hz) (FIG. 6), the left posterior hippocampus is clearly the most prominent region selected in the high frequency band (70-110 Hz) (FIG. 7). Brain areas that iEEG was recorded from: TP—temporal pole, AMY—amygdala, AHC—anterior hippocampus, PHC—posterior hippocampus, AINS—anterior insula, PARIN—parietal insula, AOF—anterior orbital frontal, POF—posterior orbital frontal. In the remaining patients, brain sites in the assessed seizure onset zone also generally coincided with the brain sites exhibiting largest percentage of time (POT) exhibiting the maximum total effective inflow (TEI) in the high frequency recordings during period Π (see Table 1).

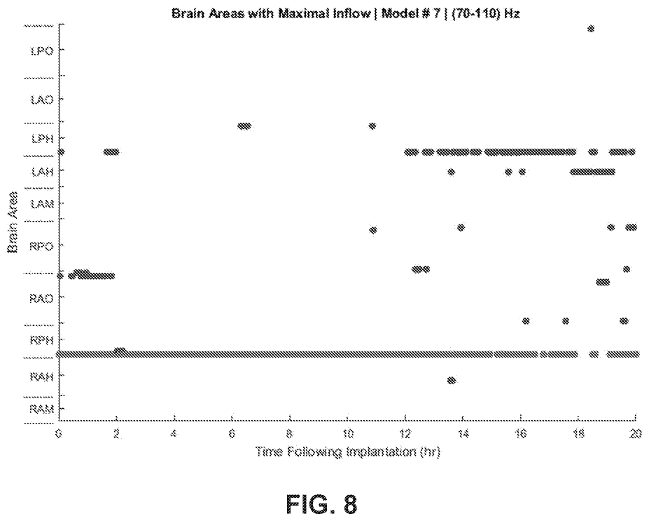

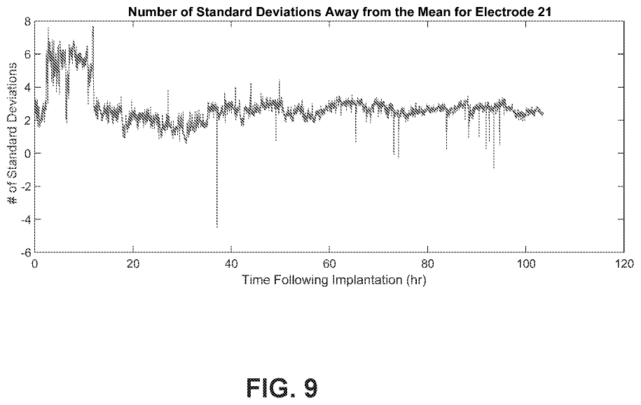

FIG. 8 shows brain sights that exhibited maximum POT per hour over time. Brain site 21 (PHC), the epileptogenic focus, exhibited maximum POT in most 1 hour intervals, especially in the beginning of the recording (period Π). FIG. 9 shows the statistical significance of the identified pathological brain site 21 (focus) over time. It is clear that the brain site 21 (identified as the focal area) is statistically (up to 6 standard deviations) an outlier when its effective inflow of information (EFI) is compared to the EFIs of the other sites in the first hours of the recording (Π period).

From the above description, those skilled in the art will perceive improvements, changes and modifications. Such improvements, changes and modifications are within the skill of one in the art and are intended to be covered by the appended claims.

What is claimed is:

1. A method comprising:
receiving, by a system comprising a processor, neurophysiological time series signals from two or more areas of a brain of an anesthetized subject, wherein the anesthetized subject is in a maintenance period of anesthesia and/or a post-emergence period of anesthesia;
estimating from the signals, by the system, a percentage of time (POT) each of the two or more areas of the brain of the subject exhibits a maximum total effective inflow (TEI) of information; and
localizing, by the system, one or more focal areas of abnormal brain interactions based on the POT of the areas exhibiting maximum TEI.

2. The method of claim 1, wherein each of the signals comprise time series data corresponding to the two or more areas of the brain of the subject.

3. The method of claim 1, wherein the signals are recorded from the brain of the subject twelve hours or less after administration of an anesthetic agent.

4. The method of claim 1, wherein a treatment plan is developed for the abnormal brain interactions based on the one or more focal areas, wherein the treatment plan comprises at least one of surgery and neuromodulation.

5. The method of claim 1, wherein the estimating further comprises:
determining an information inflow corresponding to each of the two or more areas of the brain based on an analysis of the signals, wherein the information inflow reflects a flow of information to one brain area from at least one other brain area;
comparing the information inflow corresponding to each brain area to determine the maximum TEI over segments of the period of time of recording; and
determining the POT that each of the at least two areas exhibits the maximum TEI over time.

6. The method of claim 5, wherein the determining the information inflow comprises:
fitting a model of order p to successive segments of the signals; and
determining a generalized partial directed coherence (GPDC) from a first brain signal to a second brain signal or from the second brain signal to the first brain signal in a frequency band.

7. The method of claim 6, wherein the model is a vector autoregressive model (VAR) or a multivariate autoregressive model (MVAR).

8. The method of claim 6, wherein p is a pre-determined value or an optimally determined value.

9. The method of claim 6, wherein the information inflow is determined from high frequency bands of the signals.

10. The method of claim 1, wherein the one or more focal areas are associated with at least one of an epileptic seizure disorder, a paroxysmal neurological disorder, a stroke, an autism spectrum disorder, a psychological disorder, a traumatic brain injury, an obesity disorder, an apnea disorder, a condition comprising a lack of awareness, and a neurodegenerative disease.

11. The method of claim 1, wherein the signals comprise at least one of electroencephalographic data, magnetoencephalographic data, thermal imaging data, positron emission tomography, and functional magnetic resonance imaging data.

12. A system comprising:
a non-transitory memory storing computer-executable instructions; and
a processor that executes the computer-executable instruction to at least:
receive neurophysiological time series signals from two or more areas of a brain of an anesthetized subject, wherein the anesthetized subject is in a maintenance period of anesthesia and/or a post-emergence period of anesthesia;
estimate, from the signals, a percentage of time (POT) each of the two or more areas of the brain of the subject exhibits a maximum total effective inflow (TED; and
localize one or more focal areas of abnormal brain interactions based on the POT of each of the at least two areas exhibit the maximum TEI.

13. The system of claim 12, further comprising at least one recording mechanism to record the signals, wherein the at least one recording mechanism is in communication with the non-transitory memory to deliver the signals.

14. The system of claim 13, wherein the at least one recording mechanism is configured to record at least one of electroencephalographic data, magnetoencephalographic data, thermal imaging data, positron emission tomography, and functional magnetic resonance imaging data.

15. The system of claim 12, wherein the one or more focal areas are associated with at least one of an epileptic seizure disorder, a paroxysmal neurological disorder, a stroke, an autism spectrum disorder, a psychological disorder, a traumatic brain injury, an obesity disorder, an apnea disorder, a condition comprising a lack of awareness, and a neurodegenerative disease.

16. The system of claim 12, wherein a treatment plan is developed for the abnormal brain interactions based on the identified one or more focal areas, wherein the treatment plan comprises at least one of surgery and stimulation.

17. The system of claim 12, wherein the POT is estimated by:
determining an information inflow corresponding to each of the two or more areas of the brain based on an analysis of the signals, wherein the information inflow reflects a flow of information to each of the at least two brain areas from at least one other brain area;
comparing the information inflow corresponding to each of the at least two areas to determine the maximum TEI over segments of the period of time of recording; and
determining the POT that each of the at least two areas exhibits the maximum TEI over time.

18. The system of claim 17, wherein the determining the information inflow comprises:
fitting a model of order p to successive segments of the signals; and
determining a generalized partial directed coherence (GPDC) from at least one of a first brain signal to a second brain signal or a second brain signal to a first brain signal in a frequency band.

19. The system of claim 17 wherein the model is a vector autoregressive model (VAR) or a multivariate autoregressive model (MVAR).

20. The system of claim 17, wherein the information inflow is determined from high frequency bands of the signals.
